(19) United States
(12) Patent Application Publication
(10) Pub. No.: US 2026/0335200 A1
(43) Pub. Date: Oct. 29, 2026

(54) ANTIPERSPIRANT COMPOSITIONS

(71) Applicant: Conopco, Inc., d/b/a UNILEVER, Hoboken, NJ (US)

(72) Inventors: Harriet Jade ASKEM, Leeds (GB); Emma Karen MANNION, Leeds (GB)

(73) Assignee: Conopco, Inc., d/b/a UNILEVER, Hoboken, NJ (US)

(21) Appl. No.: 19/113,906

(22) PCT Filed: Sep. 22, 2023
(86) PCT No.: PCT/EP2023/076308

§ 371 Date: Mar. 21, 2025

(30) Foreign Application Priority Data
Sep. 27, 2022 (EP) 22197945.3

(51) Int. Cl. A61K 8/06; A61K 8/04; A61K 8/20; A61K 8/26; A61K 8/34; A61K 8/44; A61K 8/893; A61Q 15/00

(52) U.S. Cl. CPC A61K 8/064; A61K 8/046; A61K 8/20; A61K 8/26; A61K 8/345; A61K 8/44; A61K 8/893; A61Q 15/00; A61K 2800/30; A61K 2800/805

(57) ABSTRACT

A water-in-oil emulsion antiperspirant aerosol composition comprising (i) an aluminium-containing antiperspirant active; (ii) at least 10% by weight of glycerol; and (iii) a nonionic emulsifier and having less than 0.1% by weight of cyclic silicone present therein.

### FIELD OF INVENTION

The present invention is concerned with antiperspirant compositions and their use to reduce perspiration on the surface of the human body. It is particularly concerned with water-in-oil (w/o) emulsion aerosol compositions comprising aluminium-containing antiperspirant active and a humectant.

### BACKGROUND

Water-in-oil emulsion antiperspirant compositions are well known in the prior art, particularly in the form of stick and gel compositions.

U.S. Pat. No. 6,911,195 (Gillette) discloses w/o emulsion gels comprising aluminium-zirconium antiperspirant salts.

WO 2009/044381 (P&G) discloses w/o emulsion sticks comprising aluminium and aluminium-zirconium antiperspirant salts activated using calcium ions.

US 2011/038823 and US 2011/038902 (Dial/Henkel) discloses w/o emulsion sticks comprising an antiperspirant active prepared by combining an aluminium-based antiperspirant active, calcium chloride and glycine.

There are also prior art publications on w/o antiperspirant aerosol compositions.

U.S. Pat. No. 4,695,451 (Colgate-Palmolive, 1987) discloses w/o emulsion aerosol antiperspirant compositions, preferably having a C3-C₄ hydrocarbon propellant present.

US 2007/292358 A1, (Emmerling, 2007) discloses storage-stable w/o emulsion aerosol antiperspirant compositions and a dispensing apparatus suitable for use therewith.

WO 2004/030641 A1 (Beiersdorf, 2004) discloses w/o emulsions, including antiperspirants compositions, sprayable as aerosols.

US 2006/051306 (Unilever, 2006) discloses w/o antiperspirant emulsion compositions comprising polymers comprising Brønsted acid groups, said compositions optionally comprising a volatile propellant and being sprayable as an aerosol.

### SUMMARY OF THE INVENTION

It is an object of the present invention to provide an emulsion antiperspirant aerosol composition that delivers both good antiperspirancy performance and good sensory properties.

It is an object of the present invention to provide an emulsion antiperspirant aerosol composition which is essential free of cyclic silicone, in particular cyclopentasiloxane.

It is a particular object of the present invention to provide an emulsion antiperspirant aerosol composition that delivers good antiperspirancy performance without causing undue drying of the skin from a sensory perspective.

It is a particular object of the present invention to provide an emulsion antiperspirant aerosol composition that delivers good antiperspirancy performance without causing undue irritation of the skin.

It is a further object of the present invention to provide a cosmetic composition which is a water-in-oil emulsion having good emulsion stability, particularly at elevated temperatures.

In a first aspect of the present invention, there is provided an emulsion antiperspirant aerosol composition comprising:
(i) an aqueous disperse phase comprising an aluminium-containing antiperspirant active and at least 10% by weight of a humectant selected from glycerol, propylene glycol and PEG oligomers of weight average molecular weight of from 180 to 420;
(ii) an oily continuous phase comprising one or more oils and having less than 0.1% by weight of cyclic silicone present therein and
(iii) a nonionic emulsifier;
wherein the aqueous disperse phase comprises at least 50% by weight of the total composition, excluding any propellant present in the composition.

In a second aspect of the present invention, there is provided a method of manufacture of an antiperspirant composition according to the first aspect of the invention.

In a third aspect of the present invention, there is provided a method of attaining an antiperspirant benefit comprising the topical application to the surface of the human body of a composition according to the first aspect of the invention.

### DETAILED DESCRIPTION

Herein, emulsion antiperspirant aerosol compositions according to the invention are w/o emulsions, as will be understand by their having an aqueous disperse phase and an oily continuous phase. The aqueous phase is dispersed within the oily continuous phase.

Herein, “emulsion stability” refers to the ability of a composition to stay homogeneous and not to separate into two or more distinct phases.

Herein, an aerosol composition is a liquid composition capable of forming an aerosol when expelled through an orifice suitable for producing an aerosol.

Herein, topical application refers to application to the skin of the human body, in particular to the underarm regions of the human body.

Herein, antiperspirant actives are materials that reduce perspiration on the skin of the human body, typically following topical application.

Herein, methods and uses should be understood to be cosmetic methods/uses, i.e. non-therapeutic methods/uses. Likewise, compositions of the invention are cosmetic compositions.

Herein, all percentages, parts, and ratios are by weight, unless otherwise indicated.

Herein, references to an amount of a material or materials refer to the total amount of material(s) of the type indicated.

Herein, references to an amount of a material are with reference to the total composition of which it is a part, unless otherwise indicated.

Herein, “application” and “applied” relate to application to the skin of the human body, in particular the underarm regions, unless the context dictates otherwise.

Herein, features expressed as “preferred” with regard to a particular aspect of the invention should be understood to be preferred with regard to each aspect of the invention.

Herein, preferred, particularly preferred and especially preferred features of the invention are particularly preferred when used in combination with other preferred, particularly preferred, and especially preferred features of the invention.

Herein, “ambient conditions” refer to 20 °C and 1 atmosphere pressure, unless otherwise indicated.

Herein, the word “comprising” is intended to mean “including” but not necessarily “consisting of” or “composed of.” In other words, the listed steps or options need not be exhaustive.

Herein, except in the examples or where otherwise explicitly indicated, all numbers in this description and claims indicating amounts of material, physical properties of materials and/or use are to be understood as modified by the word “about”.

Herein, numerical ranges expressed in the format “from x to y” are understood to include x and y. When for a specific feature multiple preferred ranges are described in the format “from x to y”, it is understood that all ranges combining the different endpoints are also contemplated.

The aqueous disperse phase comprises water and an aluminium-containing antiperspirant active. It also comprises at least 10% by weight of a humectant selected from glycerol, propylene glycol and PEG oligomers of weight average molecular weight of from 180 to 420.

The aluminium-containing antiperspirant active used in the present invention preferably has aluminium to chloride molar ratio of from 1.25:1 to 1.82:1 and preferably 1.54:1 to 1.82:1.

The aluminium-containing antiperspirant active is preferably an aluminium sesquichlorohydrate (ASCH) salt having the chemical formula Al₂OH_{4.4}Cl_{1.6} to Al₂OH_{4.9}Cl_{1.1}. Most commercial ASCH samples are of chemical formula Al₂OH_{4.7}Cl_{1.3} to Al₂OH_{4.9}Cl_{1.1} and it is preferred to have ASCH salts of this formula.

In preferred embodiments, the composition also comprises a water-soluble calcium salt and an amino acid, particular when the antiperspirant active is ASCH. It is particularly preferred that the ASCH is activated by the water-soluble calcium salt and an amino acid (vide infra).

The water-soluble calcium salt is preferably used at such a level that the molar ratio of calcium to aluminium is at least 1:40, more preferably at least 1:30 and most preferably at least 1:20. It is not advantageous to have the calcium concentration in excess of the aluminium concentration; indeed, it is preferred that the calcium concentration is no more than half that of the aluminium concentration and more preferred that it is no more than a fifth of said concentration. For the preferred molar ratios of calcium to aluminium of at least 1:40 and at least 1:20, it is independently preferred that this ratio is no greater than 1:2 and more preferred that it is no greater than 1:5.

In particularly preferred embodiments, the molar ratio of calcium to aluminium is at least 1:15 and preferably no greater than 1:5 and in especially preferred embodiments it is at least 1:10 and preferably no greater than 1:5.

A preferred water soluble calcium salt for use in the present invention is calcium chloride.

Herein, references to molar amounts and ratios of “aluminium” are calculated on the basis of mono-nuclear aluminium, but include aluminium present in poly-nuclear species; indeed, most of the aluminium in the salts of relevance is present in poly-nuclear species.

The amino acid is preferably used at such a level that the molar ratio of amino acid to aluminium is at least 1:20, more preferably at least 1:10 and most preferably at least 1:5. It is not advantageous to have the amino acid concentration in excess of the aluminium concentration; hence, the molar amino acid to aluminium is preferably from 1:20 to 1:1, more preferably from 1:10 to 1:1 and most preferably from 1:5 to 1:1.

In particularly preferred embodiments, the molar ratio of amino acid to aluminium is at least 1:4 and preferably no greater than 1:1 and in especially preferred embodiments it is at least 1:3 and preferably no greater than 1:1.

In preferred embodiments, the molar ratio of calcium to aluminium is at least 1:40 and the molar ratio of amino acid to aluminium is at least 1:20. In further preferred embodiments the molar ratio of calcium to aluminium is at least 1:20 and the molar ratio of amino acid to aluminium is at least 1:10. In particularly preferred embodiments the molar ratio of calcium to aluminium is from 1:20 to 1:5 and the molar ratio of amino acid to aluminium is from 1:10 to 1:1.

The above indicated preferences for calcium to aluminium molar ratio and/or amino acid to aluminium molar ratio lead to compositions of higher Band III content (vide infra) and, in general, higher antiperspirancy performance. It will be noted that higher Band III content is generally indicative of higher antiperspirancy performance.

Preferred amino acids for use in the present invention are glycine, alanine, valine and proline. A particularly preferred amino acid for use in the present invention is glycine.

The presence of the water-soluble calcium salt and glycine can lead to “activation” of the antiperspirant salt, by which is meant an increase in its Band Ill content (vide infra). In preferred embodiments, an aqueous solution of the antiperspirant salt is heated with the water-soluble calcium salt and glycine to achieve activation.

The content of “Band III material” in an antiperspirant salt may be determined by SEC (Size Exclusion Chromatography) analysis. The SEC technique is well known in the art and is described in further detail in U.S. Pat. No. 4,359,456 (Gosling). The SEC band commonly referred to as Band III is designated as “Peak 4” in EP 1,104,282 B1 by Gillette.

Herein, “Band Ill content” refers to the integrated area in the Band III region of the SEC chromatograph relative to the total integrated area in all of the regions corresponding to aluminium species; that is to say, Bands I, II, III, and IV.

In preferred embodiments of the invention, compositions according to the invention have antiperspirant active having a Band III content of at least 30% and more preferably at least 50%.

The antiperspirant active is present in the total composition at a preferred level of from 2 to 15%, more preferably from 3 to 10% and most preferably from 3 to 6%. The level of antiperspirant active excludes any water-soluble calcium salt or amino acid also present in the composition and/or associated with the antiperspirant active; the same is also true w.r.t. ratios relating to the amount of antiperspirant active.

Another essential feature of the aqueous continuous phase is a humectant selected from glycerol, propylene glycol and PEG oligomers of weight average molecular weight of from 180 to 420, this humectant being present at least 10% by weight of the aqueous continuous phase. The humectant serves to enhance the sensory properties of the composition.

In preferred embodiments the humectant is glycerol.

The humectant is present in the total composition at a preferred level of from 1 to 20%, more preferably from 2 to 12% and most preferably from 3 to 10%.

In order to optimise the balance of antiperspirant efficacy and sensory benefits delivered by the compositions of the invention, the weight ratio of antiperspirant active to humectant as defined herein is preferably from 1:10 to 10:1, more preferably from 1:4 to 4:1 and most preferably from 1:2 to 3:1. These ratios are of particular importance when the antiperspirant active is ASCH and/or when the humectant is glycerol.

An essential component of the invention is the oily continuous phase in which the aqueous phase is suspended. The oily continuous phase comprises one or more oils and has less than 0.1% cyclic silicone present therein. In preferred embodiments, the oily continuous phase comprises less than 0.01% of cyclic silicone. Cyclic silicones, and cyclopentasiloxane in particular, are undesirable for multiple reasons in emulsion antiperspirant compositions. In particularly preferred compositions according to the invention, there is no cyclic silicone present.

In preferred embodiments the oily continuous phase comprises a linear dimethicone (silicone) oil at a level of at least 25% of those materials present in the oily continuous phase. Preferably, this level is at least 50%, i.e. from 50 to 100%. In certain preferred embodiments, the silicone oil comprises greater than 60% by weight of the material present in the oily continuous phase.

Herein, the term “oily” indicates the presence of a water-insoluble organic material that is liquid at 20 °C. Any material having a solubility of less than 0.1 g/100 g at 20 °C is considered insoluble.

In certain embodiments, it is preferred to include an oil, other than a fragrance oil or silicone oil that has a relatively low viscosity, by which is meant less 250 cS (mm²·s⁻¹). Such oils can improve the sensory properties of the composition on application and can lead to other benefits such as emolliency.

Suitable oils can be selected from alkyl ether oils having a boiling point of above 100 °C and especially above 150 °C, including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

Suitable oils can include one or more triglyceride oils. The triglyceride oils commonly comprise the alkyl residues of aliphatic C₇ to C₂₀ alcohols, the total number of carbon atoms being selected in conjunction with the extent of olefinic unsaturation and/or branching to enable the triglyceride to be liquid at 20 °C. One example is jojoba oil. Particularly preferably, in the triglyceride oil the alkyl residues are linear C₁₈ groups having one, two or three olefinic degrees of unsaturation, two or three being optionally conjugated, many of which are extractable from plants (or their synthetic analogues), including triglycerides of oleic acid, linoleic acid, conjugated linoleic acids, linolenic acid, petroselenic acid, ricinoleic acid, linolenelaidic acid, trans 7-octadecenoic acid, parinaric acid, pinolenic acid, punicic acid, petroselenic acid and stearidonic acid. Suitable oils can include those derived from unsaturated C₁₈ acids, including coriander seed oil, impatiens balsimina seed oil, parinarium laurinarium kernel fat oil, sabastiana brasilinensis seed oil, dehydrated castor seed oil, borage seed oil, evening primrose oil, aquilegia vulgaris oil, sunflower (seed) oil and safflower oil. Other suitable oils are obtainable from hemp, and maize corn oil. An especially preferred oil is sunflower (seed) oil.

Further suitable oils, that can also be emollient oils, comprise alkyl or alkyl-aryl ester oils having a boiling point of above 150 °C (and a melting point of below 20 °C). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C₁₂₋₁₅ alkyl benzoate, for example Finsolv TN™ or Finsolv Sun™.

The water-in-oil emulsion compositions of the present invention have a balance of aqueous phase to oil phase that is at least equal 1:1, i.e. the internal phase is of at least the same total weight than the continuous phase. The ratio of aqueous phase to oil phase is preferably from 50:50 to 90:10, more preferably from 70:30 to 85:15 and most preferably from 75:25 to 85:15.

The water-in-oil antiperspirant compositions of the present invention comprise a nonionic emulsifier.

In preferred embodiments, the nonionic emulsifier is a silicone-based emulsifier. Herein, a silicone-based emulsifier is an emulsifier comprising silicone. Such emulsifiers are particularly preferred in compositions comprising a linear dimethicone, for example at a level of at least 50% or at least 60% of those materials present in the oily continuous phase.

The emulsifier is typically introduced into the composition as part of the oily continuous phase; however, it should not be considered to be part of this phase in calculating ratios, parts and percentages based upon the amount of the oily continuous phase. In reality, much of the emulsifier sits on the interface between the disperse aqueous phase and oily continuous phase, thereby stabilising the w/o emulsion composition.

Preferred silicone-based emulsifiers are dimethicone ethoxylates/propoxylates, optionally with one or more alkyl substituents. Such emulsifiers may be silicone emulsifiers that are ethoxylated and/or propoxylated silicone ethers, optionally with one or more alkyl groups. Such emulsifiers include PEG-dimethicones, PPG-dimethicones, or PEG/PPG-dimethicone copolymers.

For reasons of emulsion stability, particularly preferred silicone-based emulsifiers are PEG-10 dimethicone and PEG/PPG-18/18 dimethicone; especially preferred is PEG-10 dimethicone.

Such preferred emulsifier systems can lead to both high antiperspirancy performance and superior storage stability, particularly at high temperatures.

The total proportion of emulsifiers in the composition, excluding any volatile propellant present therein, is preferably at least 1% and particularly at least 1.3% by weight. Preferably the emulsifiers are not present at above 7%, more preferably not more than 5% and most preferably up to 3% by weight. An especially desirable concentration range for the emulsifiers is from 1.4 to 2.9%, all of these preferred ranges being based on the composition excluding any volaille propellant present therein.

The level and choice of silicone-based emulsifier can help with the balance between emulsion stability and antiperspirant efficacy which is a key factor in formulating water-in-oil emulsion antiperspirant compositions. Too little emulsifier can lead to poor emulsion stability, whilst too much can be detrimental to antiperspirant performance.

For reasons of emulsion stability and antiperspirancy efficacy, the ratio of silicone-based emulsifier to aqueous phase is preferably from 1:99 to 10:90, more preferably from 1.5:98.5 to 7.5:92.5 and most preferably from 2.0:98.0 to 3.8 to 96.2. These ratios are of particular relevance when the aqueous phase contains from 10 to 30% of the antiperspirant active.

A propellant is a highly preferred component of compositions of the invention; however, it is not essential since aerosols can be generated by other means, such as pump spray dispensers or squeeze spray dispensers.

A preferred propellant for use herein is a volatile propellant, i.e. one having a boiling point of below 10 °C and especially one with a boiling point below 0 °C. Such materials are typically in liquefied form at the pressure within the container from which they are dispensed.

Suitable volatile propellants include trichlorofluoromethane, trichlorotrifluoroethane, difluoroethane, propane, butane, isobutane, or combinations thereof. It is especially preferred to employ liquefied hydrocarbon gases, and especially C₃ to C₆ hydrocarbons, including propane, butane, isobutane, pentane and isopentane and mixtures of two or more thereof. Of these especially preferred propellants, isobutane, isobutane/propane, butane/propane and mixtures of propane, isobutane and butane are most preferred. The amount used is typically from 5 to 95% and preferably from 30 to 90% by weight of the total composition.

When a volatile propellant is used, the ratio of propellant to base is preferably from 60:40 to 90:10, more preferably from 65:35 to 90:10 and most preferably from 70:30 to 90:10.

Herein, the “base” is composed of all of components of the composition except any propellant that may be present therein.

A preferred additional optional component for use in accordance with the present invention is a fragrance oil, sometimes alternatively called a perfume oil. The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to the same.

In some embodiments, the fragrance may be an encapsulated fragrance.

The amount of fragrance oil in the composition is commonly up to 3% advantageously is at least 0.5% and particularly from 0.8% to 2%, based on the composition excluding any volatile propellant present therein.

The compositions of the present invention are suitable for dispensing via an aerosol dispenser. Such dispensers may be pressurised by compressed gas and/or a liquified gas, both of which are to be considered volatile propellants.

### EXAMPLES

The raw materials used in the Examples were as described below and in Table 1.

TABLE 1

| Raw Material | | |
|---|---|---|
| Name | Details | Supplier |
| Antiperspirant actives | | |
| AASCH | As described above | |
| ZAG Rezal 370 | Al—Zr tetrachlorohydrate glycine complex | Summit Reheis |
| AACH-7172 | Activated aluminium chlorohydrate | Elementis |
| Liquids/Oils | | |
| Glycerol | Pricerine 9091 | Croda |
| PG | Propylene glycol | Huntsman |
| Dimethicone | Dimethicone fluid, viscosity 5 cst | Dow Corning |
| Arlamol E | PPG-15 stearyl ether | Croda |
| Finsolv TN | C12-15 alkyl benzoate | Innospec |
| Fluid AP | PPG-14 butyl ether | Ucon |
| Cetiol OE | Dicaprylyl ether | BASF |
| Cetiol Ultimate | Undecane and tridecane | BASF |
| Eutanol G | Octyldodecanol | BASF |
| Akosun | Sunflower seed oil (Helianthis Annuus) seed oil | Cargill |
| Emulsifiers | | |
| Abil EM90 | Cetyl PEG/PPG-10/1 Dimethicone | Evonik |
| Brij 72P S21 | Steareth-2 | Croda |
| Volpo S2 | Steareth-21 | Croda |
| Dowsil ™ ES-5612 | PEG-10 dimethicone | Dow |
| DC5225C | PEG/PPG-18/18 dimethicone in | Dow |
| | cyclopentasiloxane (12.5% dispersion) | |
| Dowsil ™ ES-5227 | PEG/PPG-18/18 dimethicone in dimethicone | Dow |
| | (25% dispersion) | |
| Other materials | | |
| EDTA | Disodium EDTA | Biochem |
| BHT | Butylated hydroxytoluene | Eastman |
| PC | Propylene carbonate | |
| Fragrance | Commercially available | Various |
| CAP 40 | Butane, isobutane, propane | Harp Int. |

The aluminium sesquichlorohydrate (ASCH) was pre-activated by heating with the calcium chloride, glycine and water to give an aqueous solution comprising 3.240 parts ASCH, 0.404 parts calcium chloride, 0.317 parts glycine and 8.039 parts water (indicated as “aqua” in the Examples). The pre-activating step was performed for 2 hours at 87 °C.

The Examples detailed in Table 2 were prepared by methods known in the art. The “AASCH” antiperspirant active is described with reference to its constituent parts: ASCH, calcium chloride, glycine and water (“aqua”).

TABLE 2
Water-in-Oil Antiperspirant Aerosol Compositions

| | Example: | | | | | |
|---|---|---|---|---|---|---|
| | A492 | H29 | G60 | F54 | N80 | B81 |
| Components: | Parts by Wt. | | Parts by Wt. | | Parts by Wt. | |
| ASCH | 3.240 | 3.240 | 3.24 | 4.86 | 1.874 | 1.873 |
| CaCl₂ | 0.404 | 0.404 | 0.40 | 0.60 | 0.234 | 0.234 |
| Glycine | 0.317 | 0.317 | 0.32 | 0.48 | 0.183 | 0.183 |
| Aqua | 8.039 | 8.039 | 8.04 | 12.06 | 4.649 | 4.648 |
| Water | — | — | — | — | 1.51 | 1.000 |
| Glycerol | 4.000 | 4.000 | 4.00 | 6.00 | 1.30 | 1.000 |
| Dimethicone | 2.254 | 2.550 | 2.814 | 4.221 | 2.35 | 1.837 |
| Dowsil ™ | 0.600 | 0.400 | 0.60 | 0.90 | 0.20 | — |
| ES-5612 | | | | | | |
| DC5225C | — | — | — | — | — | 1.625 |
| Akosun | 0.154 | 0.150 | 0.154 | 0.231 | 0.10 | — |
| Fragrance | 0.992 | 0.900 | 0.432 | 0.648 | 0.60 | 0.600 |
| Propellant | 80.00 | 80.00 | 80.00 | 70.00 | 87.00 | 87.00 |
| Total | 100.00 | 100.00 | 100.00 | 100.00 | 100.00 | 100.00 |

A “Sweat weight reduction” (SWR) test was performed for Example A492 using a test panel of 33 female volunteers. Test operators applied Example A492 (2 second spray; 1.7-2.0 g) to one axilla and a non-antiperspirant deodorant body spray to the other axilla of each panellist. This was done once each day for three days. After the third application, panellists were requested not to wash under their arms for the following 24 hours.

24 hours after the third and final product application, the panellists were induced to sweat in a hot-room at 40 °C (±2 °C) and 40% (±5%) relative humidity, for 40 minutes. After this period, the panellists left the hot-room and their axillae were carefully wiped dry. Pre-weighed cotton pads were then applied to each axilla of each panellist and the panellists re-entered the hot-room for a further 20 minutes. Following this period, the pads were removed and re-weighed, enabling the weight of sweat generated to be calculated.

In a further test, the same procedure was followed, with the modification that it was 48 hours after the third and final product application that the panellists were induced to sweat in the hot-room.

The sweat weight reduction (SWR) for each panellist was calculated as a percentage (% SWR) and the mean % SWR was calculated according to the method described by Murphy and Levine in “Analysis of Antiperspirant Efficacy Results”, J. Soc. Cosmetic Chemists, 1991 (May), 42, 167-197.

The SWR result obtained for Example A492 was 23% after 24 hours and 17% after 48 hours. Both of these results were significantly different to that from the Control at the 95% level.

A “Sweat weight reduction” (SWR) test was performed for Example H29 using a test panel of 33 female volunteers and a method analogous to that used in the test described above. A SWR result of 29% was obtained after 48 hours, this result being significantly different to that from the Control at the 95% level. This represents a good result for a water-in-oil emulsion AP aerosol. It is also noteworthy that this result is better than that found for Example A491 after 48 hours. This is presumed to be due to the lower level of emulsifier in Example H29, although there was a procedural difference between the SWR test on Example A491 and that on Example H29, making this a less than perfect comparison. The 0.4% emulsifier in Example H29 equates to an emulsifier to aqueous phase ratio of 2.4:97.6.

Sensory tests were also performed on selected compositions according to the invention. Two compositions not according to the invention were also involved in these tests and these are detailed in Table 3. The Control composition indicated was a suspension antiperspirant aerosol composition having excellent skin care credentials, resulting from no less than four skin benefit oils present therein.

A first sensory test was performed using Examples F54 and G60 from Table 2 and the Control composition from Table 3. The results are shown in Tables 4 to 6.

The test was a single centre, balanced incomplete block design, randomised, double-blind axillary study with three study products conducted with healthy females aged 18-65. The study was divided into two cohorts of subjects staggered by 8 days and consisted of two phases: Provocation and Recovery.

TABLE 3

Compositions

| | |
|---|---|
| ProCom | Control |

| | |
|---|---|
| Components | Parts by Wt. |

| | | |
|---|---|---|
| Water | 71.65 | — |
| ZAG Rezal 370 | 18.40 | — |
| AACH-7172 | — | 3.4 |
| Aqua | 1.60 | 0.6 |
| EDTA | 0.10 | — |
| BHT | 0.05 | 0.1 |
| Arlamol E | 4.00 | — |
| Fluid AP | — | 2.89 |
| Finsolv TN | — | 0.5 |
| Eutanol G | — | 0.11 |
| Akosun | — | 0.52 |
| Volpo S2 | 2.60 | — |
| Brij 72P S21 | 0.60 | — |
| Fragrance | 1.00 | 0.65 |
| PC | — | 0.05 |
| Propellant | — | 87 |

During the Provocation phase (days 1 to 7) 67 subjects (split across 2 cohorts) provoked irritation in the axilla by daily shaving, using a razor and unperfumed soap, and exaggerated use of an antiperspirant roll-on composition (“ProCom” in Table 2) four times daily (recommended dose 250 mg per application). During this phase, underarm washing was restricted to twice daily (once each morning and once each evening) just prior to the first and fourth product applications each day, except on test centre visits when subjects washed at home at least 2 hours before their appointment and applied their first application supervised on-site.

An Expert Assessor visually assessed erythema and dryness at Days 1, 3 and 6 and subjects were asked to change their application or shaving habits depending on their individual skin response, in order to generate a sufficient and acceptable level of irritation.

Subjects who achieved an acceptable level of irritation proceeded to the Recovery phase (days 8 to 15). Subjects applied two of the three test products (one to each axilla according to the randomisation) twice daily (target dose 2 sec. per application (expected dose was 1.5-1.7 g for F54 and G60 and 1.8-2.0 g for the Control). Subjects limited their shaving to three times during the week (on the evenings of Days 8, 11 and 13), 36 (±/−4) hours prior to their next visit. The Expert Assessor visually assessed dryness and erythema at all visit days (Days 1, 3, 6, 8, 10, 13 and 15). The results from days 10, 13 and 15 are summarised in Tables 4 and 5, respectively. During this phase underarm washing was restricted to once daily only—each morning just prior to the first product application of the day (except on test centre visits when subjects washed at home at least 2 hours before their appointment and applied their first application supervised on-site). On days when subjects shave (8, 11 & 13) they washed twice, with the second wash being conducted at the same time as the evening shave.

In this study the assessment of visual skin dryness was performed according to the Clinical Guideline for Visual Dryness Assessment (GD-CP-010, V1 15 Feb. 2019); however, the 20-minute acclimatisation period prior to assessment required by the guideline was not implemented in this study.

In addition to the visual assessments, instrumental measurements of underarm skin hydration were recorded at baseline (Day 1), at the end provocation/start of recovery (Day 8) and at the end of recovery (Day 15) using a Courage and Khazaka Multi-Probe Adaptor Corneometer, MPA 6. The Day 15 results are presented in Table 5. Measurements were made in the fossa and the vault. Combined results for fossa and vault are also presented.

These results shown in Table 4 are indicative of reduced visual dryness following application of compositions of the invention, compared with application of the Control; all six results for Examples F54 and G60 are numerically less than the corresponding result for the Control. In addition, Example G60 showed significantly less dryness on days 10 and 13 compared with the Control at a greater than 95% level of confidence.

TABLE 4
Visual Dryness Results

| | | Visual dryness scores | | |
|---|---|---|---|---|
| Assessed on . . . | Example | n | Mean* | Std. Dev. |
| Day 10 | F54 | 39 | 0.615 | 0.068 |
| | G60 | 39 | 0.512 | 0.068 |
| | Control | 38 | 0.711 | 0.068 |
| Day 13 | F54 | 39 | 0.446 | 0.063 |
| | G60 | 40 | 0.381 | 0.062 |
| | Control | 39 | 0.570 | 0.068 |
| Day 15 | F54 | 40 | 0.249 | 0.050 |
| | G60 | 40 | 0.236 | 0.050 |
| | Control | 40 | 0.352 | 0.050 |

*Derived by correcting raw data measurements for differing levels of visual dryness after the provocation stage, i.e. “baseline adjusted”. This allows for a more robust statistical analysis of the data and better comparison between product performances.

These results shown in Table 5 (see below) are indicative of reduced visual erythema following application of compositions of the invention, compared with application of the Control; all six results for Examples F54 and G60 are numerically less than the corresponding result for the Control. In addition, Example G60 showed significantly less dryness on day 15 compared with the Control at a greater than 95% level of confidence, with the performance baseline adjusted as described above.

TABLE 5
Visual Erythema Results

| | | Visual erythema scores | | |
|---|---|---|---|---|
| Assessed on . . . | Example | n | Mean | Std. Dev. |
| Day 10 | F54 | 39 | 1.09 | 0.47 |
| | G60 | 39 | 1.05 | 0.43 |
| | Control | 38 | 1.12 | 0.34 |
| Day 13 | F54 | 39 | 0.74 | 0.34 |
| | G60 | 40 | 0.74 | 0.36 |
| | Control | 39 | 0.81 | 0.36 |
| Day 15 | F54 | 40 | 0.56 | 0.28 |
| | G60 | 40 | 0.51 | 0.40 |
| | Control | 40 | 0.69 | 0.43 |

TABLE 6
Hydration Results on Day 15

| | | Hydration scores | | |
|---|---|---|---|---|
| Assessed in . . . | Example | n | Mean | Std. Dev. |
| Fossa | F54 | 40 | 31.62 | 5.78 |
| | G60 | 40 | 30.64 | 6.22 |
| | Control | 40 | 26.20 | 4.76 |
| Vault | F54 | 40 | 31.16 | 6.07 |
| | G60 | 40 | 30.32 | 6.45 |
| | Control | 40 | 26.00 | 5.92 |
| Whole axilla | F54 | 40 | 31.39 | 5.75 |
| | G60 | 40 | 30.48 | 6.21 |
| | Control | 40 | 26.10 | 5.08 |

These results are indicative of increased skin hydration following application of compositions of the invention, compared with application of the Control; all six results for Examples F54 and G60 are higher than the corresponding result for the Control at a >95% level of significance, with the performance baseline adjusted as described above.

A second sensory test was performed using Examples B81 and N80 from Table 2 and the Control composition from Table 3. The results are shown in Tables 7 to 10. The protocol for the second sensory test was the same as that for the first sensory test described above, mutatis mutandis and with the addition of a visual irritation assessment, made by an Expert Assessor.

TABLE 7
Visual Dryness Results

| | | Visual dryness scores | | |
|---|---|---|---|---|
| Assessed on . . . | Example | n | Mean | Std. Dev. |
| Day 10 | B81 | 37 | 0.69 | 0.57 |
| | Control | 36 | 0.86 | 0.56 |
| | N80 | 35 | 0.59 | 0.49 |
| Day 13 | B81 | 37 | 0.50 | 0.55 |
| | Control | 38 | 0.72 | 0.53 |
| | N80 | 37 | 0.46 | 0.46 |
| Day 15 | B81 | 37 | 0.31 | 0.41 |
| | Control | 38 | 0.54 | 0.44 |
| | N80 | 37 | 0.31 | 0.41 |

These results are indicative of reduced visual dryness following application of compositions of the invention, compared with application of the Control; all six results for Examples B81 and N80 are numerically less than the corresponding result for the

TABLE 8
Visual Erythema Results

| | | Visual erythema scores | | |
|---|---|---|---|---|
| Assessed on . . . | Example | n | Mean | Std. Dev. |
| Day 10 | B81 | 37 | 1.26 | 0.37 |
| | Control | 36 | 1.39 | 0.38 |
| | N80 | 35 | 1.26 | 0.43 |
| Day 13 | B81 | 37 | 0.86 | 0.38 |
| | Control | 38 | 1.00 | 0.37 |
| | N80 | 37 | 0.91 | 0.39 |
| Day 15 | B81 | 37 | 0.64 | 0.37 |
| | Control | 38 | 0.82 | 0.43 |
| | N80 | 37 | 0.70 | 0.36 |

These results are indicative of reduced visual erythema following application of compositions of the invention, compared with application of the Control; all six results for Examples B81 and N80 are numerically less than the corresponding result for the

TABLE 9
Visual Irritation Results

| | | Visual irritation scores | | |
|---|---|---|---|---|
| Assessed on . . . | Example | n | Mean | Std. Dev. |
| Day 10 | B81 | 37 | 1.30 | 0.38 |
| | Control | 36 | 1.43 | 0.36 |
| | N80 | 35 | 1.31 | 0.44 |
| Day 13 | B81 | 37 | 0.97 | 0.39 |
| | Control | 38 | 1.09 | 0.35 |
| | N80 | 37 | 0.96 | 0.38 |
| Day 15 | B81 | 37 | 0.72 | 0.34 |
| | Control | 38 | 0.88 | 0.43 |
| | N80 | 37 | 0.76 | 0.40 |

These results are indicative of reduced visual irritation following application of compositions of the invention, compared with application of the Control; all six results for Examples B81 and N80 are numerically less than the corresponding result for the

TABLE 10
Hydration Results on Day 15

| | | Hydration scores | | |
|---|---|---|---|---|
| Assessed in . . . | Example | n | Mean | Std. Dev. |
| Fossa | B81 | 37 | 30.77 | 4.48 |
| | Control | 38 | 29.45 | 5.17 |
| | N80 | 37 | 31.14 | 4.96 |
| Vault | B81 | 37 | 32.20 | 6.13 |
| | Control | 38 | 29.68 | 6.47 |
| | N80 | 37 | 31.42 | 6.56 |
| Whole Axilla | B81 | 37 | 31.44 | 4.85 |
| | Control | 38 | 29.57 | 5.48 |
| | N80 | 37 | 31.28 | 5.46 |

These results are indicative of increased skin hydration following application of compositions of the invention, compared with application of the Control; all six results for Examples B81 and N80 are numerically higher than the corresponding result for the Control.

Further Examples of base compositions are detailed detail in Table 11. The mixture of ASCH, calcium chloride, glycine and water used was as in the previous examples. These Examples were placed on storage at 25 °C and 45 °C and their stability monitored for up to 28 days-results are given at the bottom of the Table.

Please note that the DC5225C used in Example 2 was a 12.5% dispersion of PEG/PPG-18/18 dimethicone in cyclopentasiloxane, so the 12.5% level indicated equated to 1.56% emulsifier and 10.94% cyclopentasiloxane. Also, the Dowsil™ ES-5227 used in Examples 3-5 was a 25% dispersion of PEG/PPG-18/18 dimethicone in dimethicone, so the actual levels of emulsifier in these Examples was only a quarter of that indicated in the Table.

TABLE 11
Water-in-Oil Antiperspirant Aerosol Base Compositions

| | Example: | | | | | |
|---|---|---|---|---|---|---|
| | 1 | 2 | 3 | 4 | 5 | 6 |
| Components: | Parts by wt. | | | | | |
| ASCH | 53.37 | 53.37 | 60 | 60 | 60 | 60 |
| CaCl₂ | | | | | | |
| Glycine | | | | | | |
| Aqua | | | | | | |
| Glycerol | 10 | 7.69 | 10 | 10 | 20 | 20 |
| Water | 11.63 | 7.69 | 10 | 10 | — | — |
| Dimethicone | 18.12 | 14.13 | 10.61 | 8.61 | 6.61 | 12.62 |
| Dowsil ™ | 1.5 | — | — | — | — | 2 |
| ES-5612 | | | | | | |
| DC5225C | — | 12.5 | — | — | — | — |
| Dowsil ™ | — | — | 4 | 6 | 8 | — |
| ES-5227 | | | | | | |
| Akosun | 0.77 | — | 0.77 | 0.77 | 0.77 | 0.77 |
| Fragrance | 4.61 | 4.62 | 4.62 | 4.62 | 4.62 | 4.61 |
| Stability at 25 °C | Good¹ | Poor² | Partial³ | Good¹ | Partial³ | Good¹ |
| Stability at 45 °C | Partial³ | Poor² | Poor² | Poor² | Poor² | Partial³ |

¹Stable for 28 days+ (test duration).
²Phase separation within 1 day.
³Stable after 1+ days, but slowly separated over subsequent days.

These results suggest that Dowsil™ ES-5612 and Dowsil™ ES-5227 are superior emulsifiers to DC5225C in emulsions of this type. With the Dowsil™ ES-5227 and DC5225C emulsifier raw materials delivering essentially the same level of the same emulsifier (PEG/PPG-18/18 dimethicone), the comparison between Example 2 and

Example 4 suggests that the superior stability of Example 4 is related to its lack of cyclopentasiloxane, although there are other differences.

In addition, Dowsil™ ES-5612 seems to be a superior emulsifier to Dowsil™ ES-5227 (note Example 5 vs. 6 at 25 °C and Example 1 vs. 3 at 45 °C.

Additional Examples of Table 12 were of acceptable stability and were capable of being formulated into an aerosol with CAP 40 at a base to propellant ratio of 10:90 or 20:80. These Examples were prepared as described for earlier Examples.

TABLE 12
Water-in-Oil Antiperspirant Aerosol Base Compositions

Example:

| | |
|---|---|
| 7 | 8 |

| | |
|---|---|
| Components: | Parts by wt. |

| | | |
|---|---|---|
| ASCH | 44.62 | 44.62 |
| CaCl₂ | | |
| Glycine | | |
| Aqua | | |
| Glycerol | 15.38 | 15.38 |
| Finsolv TN | 9.33 | 9.33 |
| Dimethicone | 9.33 | 13.37 |
| Abil EM90 | 1.92 | 1.92 |
| Akosun | 15.38 | 15.38 |
| Fragrance | 4.04 | — |

1. An emulsion antiperspirant aerosol composition comprising:
(i) an aqueous disperse phase comprising an aluminium-containing antiperspirant active and at least 10% by weight of glycerol present in the aqueous disperse phase;
(ii) an oily continuous phase comprising one or more oils and having less than 0.1% by weight of cyclic silicone present in the oily continuous phase;
(iii) a nonionic emulsifier; and
(iv) a volatile propellant;
wherein the aqueous disperse phase comprises at least 50% by weight of the total composition, excluding the propellant present in the composition; wherein the nonionic emulsifier is a PEG-dimethicone.

2. The emulsion antiperspirant composition according to claim 1, characterised in that the aqueous disperse phase of the emulsion comprises at least 20% by weight of glycerol.

3. The emulsion antiperspirant composition according to claim 1, comprising a water-soluble calcium salt and an amino acid.

4. The emulsion antiperspirant composition according to claim 3, wherein the water-soluble calcium salt is calcium chloride, and the amino acid is glycine.

5. The emulsion antiperspirant composition according to claim 1, wherein the aluminium-containing antiperspirant active is of formula Al₂OH_{4.7}Cl_{1.3} to Al₂OH_{4.9}Cl_{1.1}.

6. The emulsion antiperspirant composition according to claim 1, wherein the aluminium-containing antiperspirant active has a Band III content measured by SEC of greater than 30%.

7. The emulsion antiperspirant composition according to claim 1 wherein the antiperspirant salt is heated with a water-soluble calcium salt and glycine to achieve activation.

8. The emulsion antiperspirant composition according to claim 1, wherein the volatile propellant is a liquified gas.

9. The emulsion antiperspirant composition according to claim 1, wherein the antiperspirant active to humectant is present in a weight ratio of from 1:4 to 4:1.

10. The antiperspirant composition according to claim 1, wherein the oily continuous phase comprises at least 25% by weight of linear dimethicone fluid.

11. The antiperspirant composition according to claim 9, wherein the oily continuous phase comprises at least 50% by weight of linear dimethicone fluid.

12. (canceled)

13. (canceled)

14. The antiperspirant composition according to claim 1, comprising PEG-10 dimethicone as an emulsifier.

15. A method of attaining an antiperspirant benefit comprising the topical application to the surface of the human body of a composition according to claim 1.

16. The emulsion antiperspirant composition according to claim 1, wherein the aluminium-containing antiperspirant active has a Band III content measured by SEC of greater than 50%.

17. The emulsion antiperspirant composition according to claim 1, wherein the aluminium-containing antiperspirant active is present in the total composition from 2 to 15% by weight of the composition.

18. The emulsion antiperspirant composition according to claim 1, wherein the antiperspirant active to humectant is present in a weight ratio of from 1:2 to 3:1.

19. The emulsion antiperspirant composition according to claim 1, wherein the oily continuous phase has less than 0.01% by weight of cyclic silicone present in the oily continuous phase.

20. The emulsion antiperspirant composition according to claim 1, wherein the composition has an aqueous phase to oil phase that is present in a weight ratio from 50:50 to 90:10.

21. The emulsion antiperspirant composition according to claim 1, wherein the composition comprises a ratio of volatile propellant to base ranging from 60:40 to 90:10.
